(19) United States
(12) Patent Application Publication
(10) Pub. No.: US 2024/0123405 A1
(43) Pub. Date: Apr. 18, 2024

(54) LIQUID COLUMN SEPARATION DEVICE, SYSTEM, AND METHOD

(71) Applicant: Hitachi High-Tech Corporation, Tokyo (JP)

(72) Inventors: Ruochi HSU, Tokyo (JP); Kei TAKENAKA, Tokyo (JP); Michiru FUJIOKA, Tokyo (JP); Nobuhiro TSUKADA, Tokyo (JP); Yohei HANAZAKI, Tokyo (JP); Nobuaki YOSHIOKA, Tokyo (JP)

(21) Appl. No.: 18/275,882

(22) PCT Filed: Feb. 17, 2021
(86) PCT No.: PCT/JP2021/005855

§ 371 Date: Aug. 4, 2023

(51) Int. Cl. B01D 61/42; B01D 71/02

(52) U.S. Cl. CPC B01D 61/42; B01D 71/0213; B01D 2221/10; B01D 2259/80; B01D 2325/36; B01D 2325/38

(57) ABSTRACT

A solid type nano-pore sequencer is arrayed easily. In a liquid column separation device including first and second base materials, a liquid feeding unit supplies fluids to a gap between the two base materials. Hydrophilic and hydrophobic regions are disposed on a surface of the second base material, and the hydrophilic region is disposed to oppose a predetermined position of the first base material where a substance to be measured is made to pass through. Also, a flow passage space includes at least one inlet and outlet through which fluids can flow in and out. A representative length of the hydrophilic region is made larger than a distance between the first and second base materials, and thereby a liquid column capable of connecting the first base material and the second base material is formed by making two or more immiscible fluids to flow by the liquid feed unit.

### TECHNICAL FIELD

The present invention relates to a liquid column forming method, particularly a liquid column separation device, system, and method which are technologies related to biological measurement.

### BACKGROUND ART

As a new technology in a biological sample analyzing device sequence analyzing a biological molecule such as DNA (deoxyribonucleic acid) and protein, development of a nano-pore DNA sequencer utilizing a pore having a size of the same degree as a biological molecule is promoted.

A nano-pore DNA sequencer is configured of a membrane and chambers, the membrane having a pore, the chambers including electrodes disposed on/below the membrane. A nano-pore that is a pore on the membrane is one and only flow passage connecting the chambers separated into two layers by the membrane. When voltage is applied by the electrodes in this configuration, since such electric field that a line of electric force passes the pore is generated within the chamber, a biological molecule receives force from the electric field, and passes the nano-pore. Since change in impedance derived from the structure of the biological molecule occurs when the biological molecule passes the nano-pore, by measuring change in a value of a current flowing between both electrodes, identification of the structure of the biological molecule is enabled.

In order to improve the throughput of reading of this nano-pore DNA sequencer, it is important to execute measurement in parallel in plural nano-pores. Therefore, it is required to electrically insulate respective nano-pores from each other, however it is extremely difficult to miniaturize a structure including a separation wall electrically insulating the nano-pores from each other as well as circumferential configurations such as electrodes and wiring.

Therefore, the present inventors are studying a device electrically insulating respective nano-pores from each other by making a substrate including an electrode oppose a membrane including a nano-pore, and configuring a column-like liquid droplet (will be hereinafter referred to as a liquid column) between the nano-pore and the electrode instead of the chamber.

As a technology for producing a minute liquid droplet within a device, in Patent Literature 1, in a device for analyzing a biological molecule, there is disclosed a method for arranging plural minute liquid droplets in array within a device. The device of Patent Literature 1 is a device for observing one molecule of a fluorescent dye. The present device has a structure that a second substrate is made to oppose in parallel a first substrate that includes plural minute hydrophilic wells in the hydrophobic surface. When an oil-based sealing liquid is fed after feeding a sample liquid including a fluorescent dye between the substrates, although the gap between the substrates is filled with the oil-based sealing liquid, a sample remains within the plural wells, and therefore a well on the first substrate becomes an independent reaction chamber. When such a sample liquid is used that the number of the fluorescent dye molecule entering one reaction chamber becomes one or less, by counting the number of the reaction chamber that obtained a signal, the concentration of the detected molecule can be checked precisely.

In Patent Literature 2, there is disclosed a device capable of operating a liquid droplet sandwiched between two substrates utilizing electro-wetting that is a phenomenon that wettability of the surface of an electrode changes when electric potential is applied to the electrode. Although the substrate side where the electrode is disposed is of a water-repellent film, a hydrophilic film is disposed in the opposing substrate, and the liquid droplet can be kept at a potion of the hydrophilic film.

### CITATION LIST

### Patent Literature

Patent Literature 1: Japanese Unexamined Patent Application Publication No. 2018-121634
Patent Literature 2: Japanese Unexamined Patent Application Publication (Translation of PCT Application) No. 2016-539343

### SUMMARY OF INVENTION

### Technical Problem

As described above, the present inventors are studying a device electrically insulating respective nano-pores from each other by configuring a liquid droplet between the nano-pore and the electrode. However, with respect to the methods having been reported so far, study on the condition for forming a liquid column within the device is insufficient. With respect to Patent Literature 1, since observation of one molecule of the fluorescent dye is the object, it is required that the sample liquid including the fluorescent dye does not remain in a position other than the reaction chamber that is the observation area. When the sample liquid remains in a position other than the reaction chamber, since the fluorescence from the sample liquid remaining in a position other than the reaction chamber becomes the background light when one molecule of the fluorescent dye within the reaction chamber is observed, observation of one molecule of the fluorescent dye is hampered. Therefore, it is not preferable that the sample liquid comes to have a liquid column shape being in contact with the substrate including the well and the opposing substrate. Accordingly, it is necessary to study a condition for preventing the liquid column shape, such condition is not mentioned in Patent Literature 1. Also, in a device where the sample liquid remains only in the well, since the liquid droplet cannot be in contact with the nano-pore or the electrode, the liquid droplet cannot be used for a nano-pore DNA sequencer.

Also, according to the device of Patent Literature 2 utilizing electro-wetting, since it is required to arrange a number of electrodes and wiring, complicated processes are required. Furthermore, since it is required to apply potential to the electrode within the device for moving the liquid droplet, it is not considered to be suitable to a nano-pore DNA sequencer that measures the change of the minute current.

The object of the present invention is to provide a liquid column separation device, system, and method solving such problems as described above and forming a liquid column array of high density.

### Solution to Problem

In order to achieve the above object, this invention provides a liquid column separation device which comprises: a first base material; a second base material disposed to oppose the first base material at a predetermined distance; and a liquid feed unit feeding two or more fluids to a gap between the first base material and the second base material. In the liquid column separation device, a surface of the first base material opposing the second base material has a pattern of arraying a plurality of hydrophilic regions having hydrophilicity in a hydrophobic region having hydrophobicity, representative length of the hydrophilic region is larger than the predetermined distance, and a liquid column that is in contact with the first base material and the second base material is produced by making two or more immiscible fluids flow between the first base material and the second base material by the liquid feed unit.

Further, in order to achieve the above object, this invention provides a liquid column separation system, which comprises: a liquid column separation device that includes a first base material, a second base material disposed to oppose the first base material at a predetermined distance, and a liquid feed unit feeding two or more fluids to a gap between the first base material and the second base material, a surface of the first base material opposing the second base material having a pattern of arraying a plurality of hydrophilic regions having hydrophilicity in a hydrophobic region having hydrophobicity, representative length of the hydrophilic region being larger than the predetermined distance, a liquid column that is in contact with the first base material and the second base material being produced by making two or more immiscible fluids flow between the first base material and the second base material by the liquid feed unit, the first base material including an electrically independent first electrode in the hydrophilic region, the second base material including a membrane that includes a nano-pore that is in contact with the liquid column; a chamber including an electrolyte solution that is in contact with the membrane; a second electrode that is in contact with the chamber; a measuring unit connected to the second electrode; and a control unit controlling voltage applied to both electrodes according to a measurement result of the measuring unit. In the liquid column separation system, a biological molecule is introduced to the liquid column and is made to pass through the nano-pore, temporal change of ion current flowing between the both electrodes is measured, thereby passing through of the biological molecule is detected, and a structural characteristic of the biological molecule is analyzed.

Still further, in order to achieve the above object, this invention provides a liquid column separation method of producing a liquid column being in contact with a first base material and a second base material, by making two or more immiscible fluids flow to a gap between the first base material and the second base material of a liquid column separation device by a liquid feed unit, the liquid column separation device being configured to include: the first base material; the second base material disposed to oppose the first base material at a predetermined distance; and the liquid feed unit feeding two or more fluids to a gap between the first base material and the second base material, a surface of the first base material opposing the second base material having a pattern of arraying a plurality of hydrophilic regions having hydrophilicity in a hydrophobic region having hydrophobicity, representative length of the hydrophilic region being larger than the predetermined distance.

### Advantageous Effects of Invention

According to the present invention, by feeding two or more immiscible fluids, an independent fluid column being in contact with the second base material opposing the hydrophilic region of the first base material can be formed. Problems, configurations, and effects other than those described above will be clarified by explanation of the embodiments below.

### BRIEF DESCRIPTION OF THE DRAWINGS

FIG. 1 is an explanatory drawing illustrating a configuration example of a liquid column separation device of the first embodiment.

FIG. 2 is an explanatory drawing illustrating a forming process of a liquid column related to the first embodiment.

FIG. 3 is a graph chart illustrating an analysis result of a liquid droplet and a liquid column forming condition related to the first embodiment.

FIG. 4 is a drawing illustrating a liquid droplet and a liquid column related to the first embodiment.

FIG. 5 is a drawing illustrating a modification of the liquid column separation device of the first embodiment.

FIG. 6 is a drawing illustrating a modification of the liquid column separation device of the first embodiment.

FIG. 7 is a drawing illustrating a modification of the liquid column separation device of the first embodiment having a structure of adding a resisting body in the hydrophilic region.

FIG. 8 is a drawing illustrating a process for producing a hydrophobic region on the hydrophilic region of the first base material related to the modification.

FIG. 9 is a drawing illustrating a process for producing a pillar in the second base material related to the modification. (Resist is a mixture with main components of a resin (polymer), a photosensitizer, an additive, and a solvent.)

FIG. 10 is a schematic view illustrating a configuration of a nano-pore device related to the second embodiment.

FIG. 11 is a schematic view illustrating a configuration of a nano-pore device related to the third embodiment.

### DESCRIPTION OF EMBODIMENTS

Embodiments for implementing the present invention will be hereinafter explained consecutively according to the drawings.

### First Embodiment

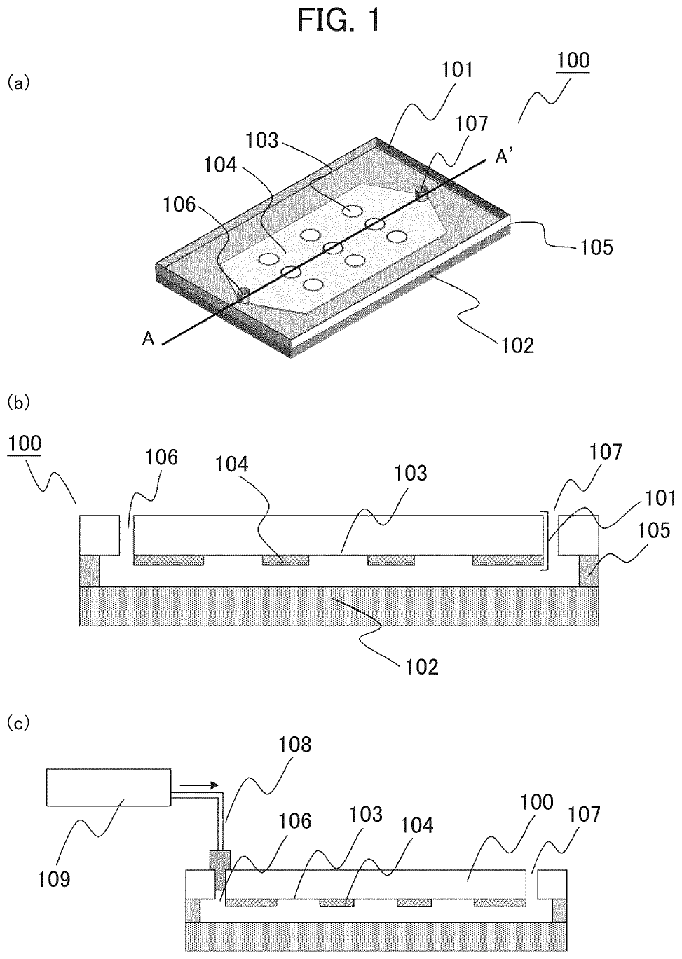

The first embodiment is an embodiment of a liquid column separation device, system, and method for separating a liquid column that connects the first base material and the second base material. First, a configuration of a liquid column separation device 100 will be explained using FIG. 1. FIG. 1 (a) is a perspective view of the liquid column separation device 100, and FIG. 1 (b) is a cross-sectional view taken along the A-A′ cross section of the perspective view. The liquid column separation device 100 is configured to sandwich a peripheral member 105 by a first base material 101 and a second base material 102, and includes a filling port 106 and a discharge port 107, the filling port 106 pouring liquid into the liquid column separation device 100, the discharge port 107 discharging the liquid.

Also, the first base material 101 includes a hydrophilic region 103 and a hydrophobic region 104. According to the present embodiment, in the first base material 101, a film of the hydrophobic region 104 is configured on the surface of the hydrophilic region 103.

The hydrophilic region 103 is of a hydrophilic solid material, and glass and oxide silicon are used for example. The hydrophobic region 104 is configured of a hydrophobic substance, namely a silane compound, a fluorine resin, and a hydrocarbon compound for example. As a fluorochemical polymer resin, an amorphous fluorochemical resin and the like can be cited for example. The amorphous fluorochemical resin has high hydrophobicity and has an advantage of low toxicity with respect to a biological sample.

In the first base material 101, a portion not covered by the hydrophobic region 104 becomes hydrophilic since the hydrophilic region 103 is exposed. The shape of the hydrophilic region 103 not covered by the hydrophobic region 104 may be a circular shape, a polygonal shape, and the like for example. According to the present embodiment, although the hydrophobic region 104 was produced on the hydrophilic region 103, the hydrophilic region may be arranged on the hydrophobic region in an opposite manner.

The second base material 102 used a hydrophilic base material, namely glass and a silicon wafer for example, the silicon wafer having an oxide film. Also, a hydrophobic material subjected to hydrophilizing surface treatment and a resin obtained by sticking hydrophilic sheets together may be used. The gap between the first base material 101 and the second base material 102 is supported by the peripheral member 105. Although the material of the peripheral member 105 is not particularly limited, a Teflon (registered trade mark) sheet, a silicone sheet, a two-sided adhesive tape, dimethylpolysiloxane (PDMS), and the like can be cited for example.

In FIG. 1 (c), an example of a method for using a liquid column device was illustrated. The filling port 106 and a liquid feed unit 109 are connected using a tube 108, and solution is fed from the liquid feed unit 109. The liquid feed unit may be a pump such as a syringe pump and a diaphragm pump, and the solution may be fed by manual operation using a syringe and a pipetter.

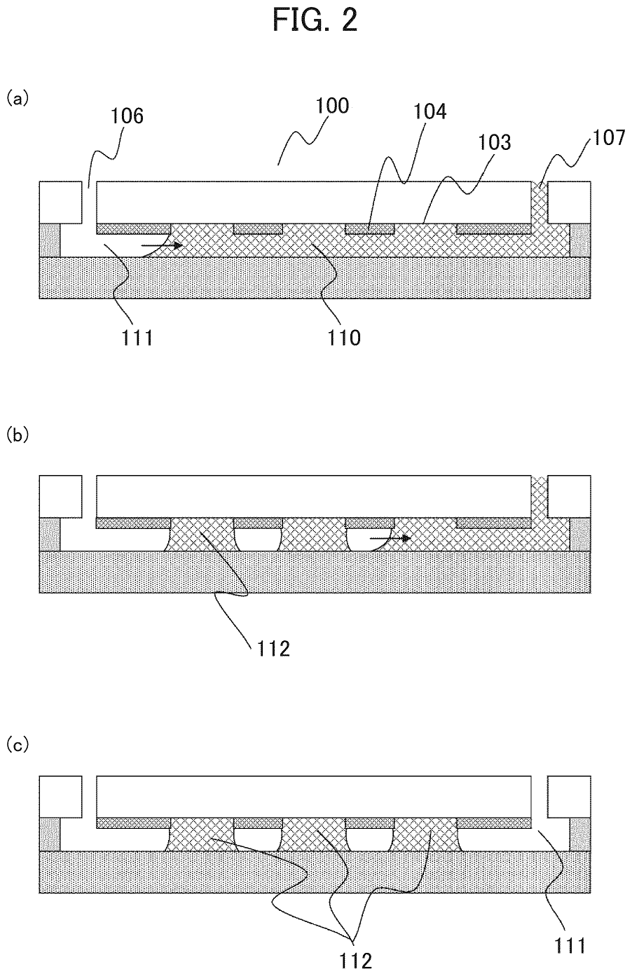

FIG. 2 illustrates a process for producing a liquid column 112 in the liquid column separation device 100 of the present embodiment. A hydrophilic first solution 110 is to be a disperse phase, and a hydrophobic second solution 111 is to be continuous phase. First, the hydrophilic first solution 110 is made to flow into the liquid column separation device 100 from the filling port 106. Excessive first solution 110 flows out through the discharge port 107. Next, the hydrophobic second solution 111 is made to flow into the liquid column separation device 100 from the filling port 106. When a condition described below is fulfilled at this time, the liquid column 112 is formed on the hydrophilic region 103 that is not covered by the hydrophobic region 104.

Although the hydrophilic liquid used in the present embodiment is an electrolyte solution containing a biological molecule that is the detection object, from the viewpoint of producing a liquid column, presence or absence of a biological molecule has no relevance, and it is important to be a hydrophilic liquid. Further, although a surfactant agent and the like may be included in the hydrophilic liquid, Tween 20 and Triron-X100 for example are preferable, Tween 20 and Triron-X100 being non-ionic surfactant agents not destructing the structure of the biological molecule. Also, a hydrophobic liquid used in the present embodiment is preferable to be a silicone oil, a mineral oil, or a fluorine-system oil. For example, as a fluorine-system liquid, Novec (registered trade mark), a polymer having a perfluoro-carbon structure, Fluorinert (registered trade mark) FC-40, Fluorinert (registered trade mark) FC-43, and the like are used.

A formation principle of the liquid column 112 described above will be explained. Within a minute flow passage, since the rate of the interfacial area to the volume becomes large, a shear force and an interfacial tension (surface force) applied to the interface of the first solution 110 that is a hydrophilic solution and the second solution 111 that is a hydrophobic solution become dominant compared to an inertia force (body force) caused by the flow velocity. A shear force is generated in the interface of two liquids, and only the first solution 110 on the hydrophilic region 103 not covered by the hydrophobic region 104 remains within the liquid column separation device 100. Next, a condition for the first solution 110 on the hydrophilic region 103 that is not covered by the hydrophobic region 104 to become the liquid column 112 will be explained.

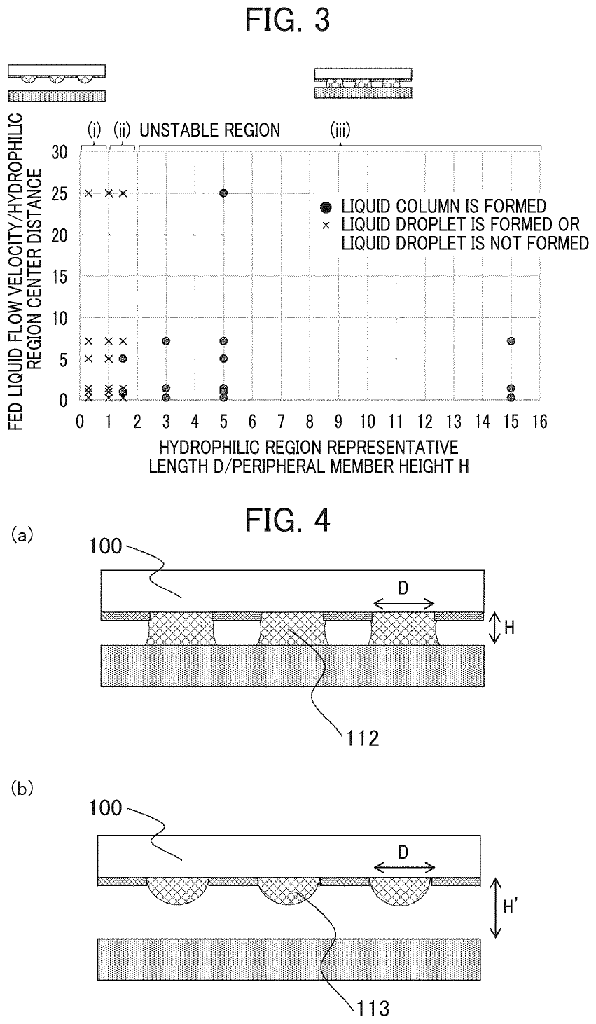

FIG. 3 is a graph chart illustrating a liquid column forming condition when the representative length of the hydrophilic region is made D and the distance between the hydrophilic surface of the first basic material and the second base material namely the height of the peripheral member 105 in the present embodiment is made H, and a fluid analysis was used for creation of the graph chart. In the graph, X-axis represents the cross-sectional aspect ratio A(=D/H), and Y-axis represents the ratio of the flow velocity of the fed liquid and the center distance (pitch) of the hydrophilic region.

As illustrated in FIG. 3, (i) a condition that the cross-sectional aspect ratio A is less than 1 is a condition where a liquid column being in contact with the base material is not formed. Next, (ii) a condition that the cross-sectional aspect ratio A is 1 or more and less than 2 is a condition where formation of a liquid column is unstable. Lastly, (iii) a condition that the cross-sectional aspect ratio A is 2 or more is a condition where a liquid column is formed stably. Therefore, the necessary condition for forming the liquid column 112 is that the cross-sectional aspect ratio A(=D/H) becomes 1 or more.

FIGS. 4 (a) and (b) are image drawings of the liquid column 112 or a liquid droplet 113 having been formed. Next, a device structure where a liquid column is more easily formed compared to the liquid column separation device 100 will be illustrated.

FIG. 5 is an example of another shape of a liquid column separation device of the present embodiment. It is probable that the hydrophilic surface 103 of the second base material 102 illustrated in FIG. 1 cannot be separated from a neighboring liquid column since the liquid spreads over the surface. Therefore, as illustrated in FIG. 5, in order to restrict the range of the liquid column being in contact with the surface of the second base material 102, a hydrophobic region 104′ is produced also on the surface of the second base material 102 as done in the first base material 101. The shape of this hydrophobic region is not limited to be invariably the same as that of the hydrophobic region of the first base material.

Also, since the sufficient condition allowing the liquid column to be formed is that the cross-sectional aspect ratio A(=D/H) is 2 or more as described above, it is necessary to make the height of the peripheral member 105 lower as the hydrophilic region is smaller. However, since the distance between two substrates becomes narrow in the case, the pressure loss when the first solution and the second solution are made to flow becomes high, and it becomes hard for the fluid to flow.

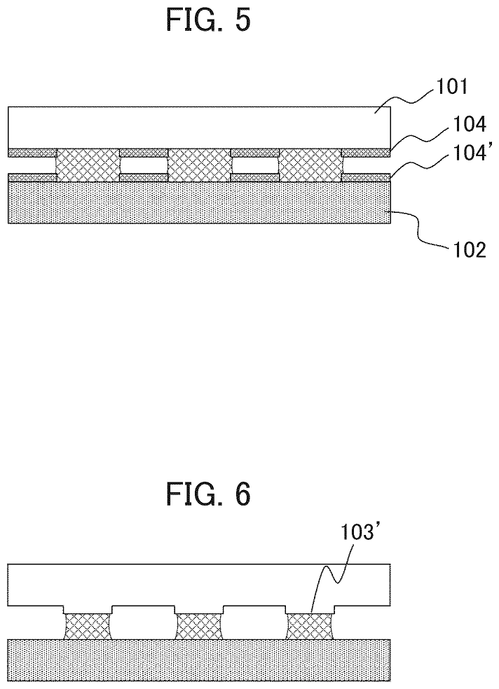

This problem can be solved by a device structure illustrated in FIG. 6. That is to say, such structure is provided that a portion of a projected structure 103′ of the base material surface is made a hydrophilic region. Although the distance between the surface of the hydrophilic region of the projected structure 103′ and the opposing base material becomes narrow, since the distance between the surface of the hydrophilic region other than the projected structure and the opposing base material can be made large, increase of the pressure loss can be suppressed. A method for producing the hydrophilic region of the projected structure 103′ is, for example, that a silicon wafer is heat-treated, a hydrophilic silicon oxide layer is formed, and the hydrophilic region can be produced using a technology such as photolithography and etching of the microfabrication technology described above. A portion where the hydrophilic silicon oxide layer is etched becomes hydrophobic since silicon is exposed.

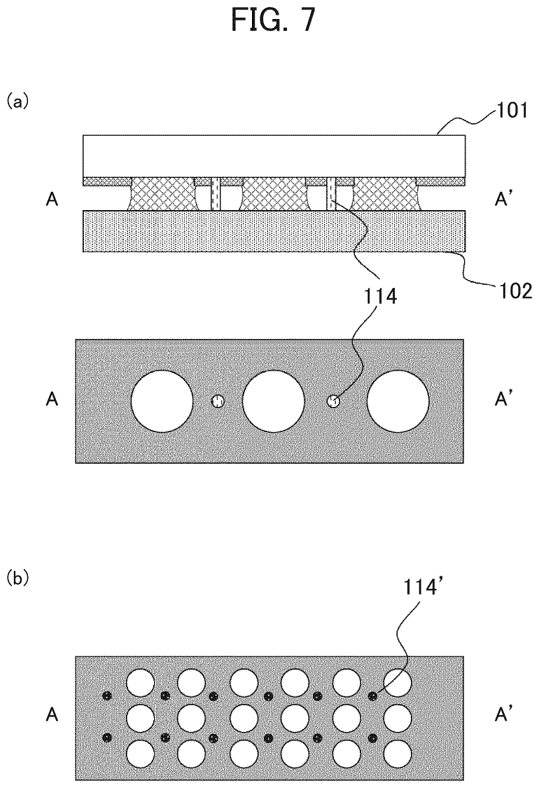

Also, since the neighboring liquid columns are possibly joined to each other, by adding a resistive element 114 between the hydrophilic regions as illustrated in FIG. 7 (a), joining of the liquid columns can be prevented. Also, within the flow passage, the flow velocity near the wall surface of the tube is slow, and the flow velocity near the center of the flow passage is fast. Therefore, as FIG. 7 (b), with combination of the first base material and the second base material, the flow velocity can be equalized by adding a resistive element 114′ between the hydrophilic regions.

With respect to the microstructure pattern of the resistive element 114′, rubber materials such as silicone rubber to begin with polydimethylsiloxane (PDMS), natural rubber, fluoro-rubber and various kinds of the plastic group can be used. Also, a transparent material such as quartz glass, float glass, calcium fluoride, silicon carbide, polymethylmethacrylate resin, and diamond or an opaque hard material including ceramics and metal such as aluminum and stainless steel can be used.

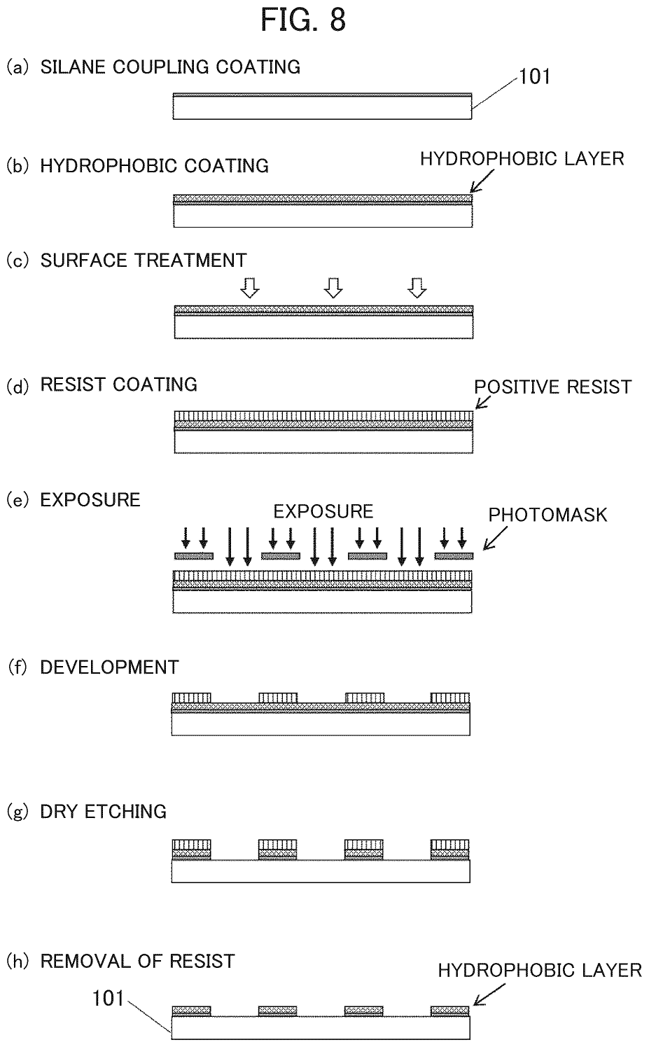

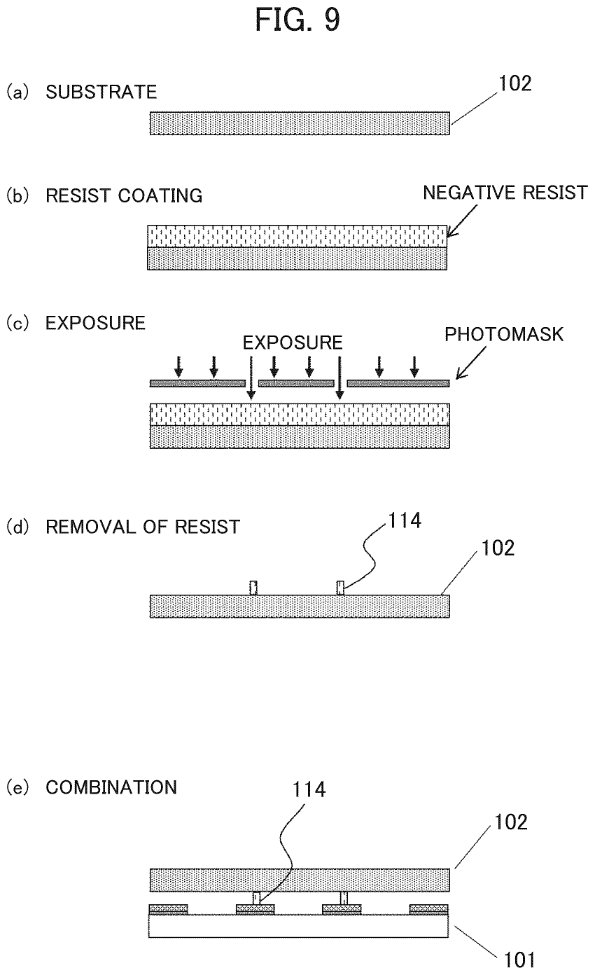

In FIG. 8 and FIG. 9, there was illustrated an example of a process for producing a hydrophobic region on the hydrophilic region of the first base material and a process for producing a pillar on the second base material related to the present embodiment. As illustrated in FIG. 8 (a) to (h), in the process for producing a hydrophobic region on the hydrophilic region of the first base material, on the first base material 101, silane coupling coating, hydrophobic coating, surface treatment, resist coating, exposure, development, dry etching, and removal of resist are executed, and a hydrophilic region is formed.

Also as illustrated in FIG. 9 (a) to (e), a resist is coated on the second base material 102 (FIG. 9 (a), (b)), removal of the resist is executed (FIG. 9 (d)) after exposure (FIG. 9 (c)) to form the resistive element 114, and by combination with the first base material 101 of FIG. 8 (FIG. 9 (e)), a resistive element can be added to the hydrophilic region illustrated in FIG. 7. Here, the resist is a mixture with main components of a resin (polymer), a photosensitizer, an additive, and a solvent.

As explained above, the present embodiment is a liquid column separation device where a first base material and a second base material are made to oppose each other, the first base material having a function for measuring a predetermined substance in the surface of the first base material, the second base material including hydrophilic and hydrophobic patterns on the surface of the second base material, a filling port for supplying fluid to a gap between the base materials and a discharge port for discharging the fluid are provided, a function for measuring a predetermined on the first base material and the hydrophilic pattern on the second base material are opposingly positioned, the representative length of the hydrophilic region is made longer than the distance between the first base material and the second base material, and thereby an independent liquid column opposing the hydrophilic region of the first base material and being in contact with the second base material can be formed.

### Second Embodiment

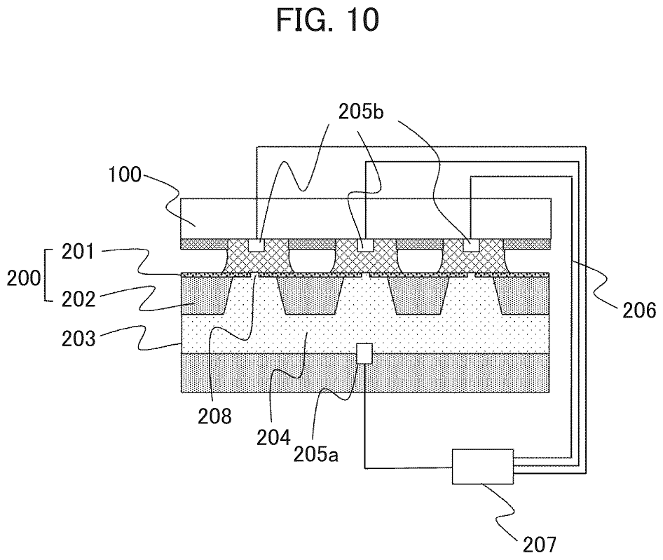

The second embodiment is an embodiment of a measuring process using a nano-pore DNA sequencer that combines a nano-pore device and a liquid column separation device. An outline of the second embodiment will be explained using FIG. 10. FIG. 10 is a schematic view illustrating a configuration of the liquid column separation device 100 and a nano-pore device 200.

The liquid column separation device 100 in the present embodiment used a device similar to that of the first embodiment, and the nano-pore device 200 was used instead of the glass substrate as the second base material. The nano-pore device 200 includes a membrane 201 and a substrate with membrane 202. It is required that the membrane 201 has a property as an insulator. Although a silicon nitride film was used in the present embodiment, a silicon oxide film, an organic substance, or a polymer material, and the like may be used. Although silver-silver chloride was used for an electrode, platinum, gold, and the like may be used. A chamber 203 is configured so that its inside can be filled with an electrolyte solution 204. With respect to a measurement device of the present embodiment, a second electrode 205a and independent first electrodes 205b are disposed, the independent first electrodes 205b being disposed at a position opposing the nano-pore device 200 and within respective hydrophilic regions of the first base material, and wiring 206 and a power supply and control/detection data acquisition unit 207 are provided. The power supply and control/detection data acquisition unit 207 includes a high-output power source, a processor, a memory, and a storage unit whose illustration is omitted.

Next, the measurement process will be explained. The measurement process includes two processes of a nano-pore formation process and a sample analysis process. First, formation of the nano-pore is executed by a following flow. The inside of the chamber 203 is filled with the electrolyte solution 204. Next, in the liquid column separation device 100, a liquid column of a water solution being in contact with the membrane 201 and the first electrode 205b is formed with a procedure shown in the first embodiment.

For the first solution 110 used in forming the liquid column and the electrolyte solution 204 within the chamber, a KCl water solution was used. Other than the KCl water solution, water solution of LiCl, NaCl, CaCl₂, MgCl₂, CsCl, and the like may be used. At this time, the first electrode 205b is in contact with the first solution 110, and the second electrode 205a is in contact with the electrolyte solution 204. When voltage is applied to the electrodes 205a and 205b, a pore (nano-pore) 208 with a nano-meter size is formed in the membrane 201.

Next, the sample analysis process will be explained. Before producing the liquid column again within the liquid column separation device 100, the inside of the liquid column separation device 100 is cleaned by a cleaning solution, and is filled with the electrolyte solution along with the chamber 203. Next, according to the procedure of the first embodiment, a liquid column of the electrolyte solution containing a biological molecule (DNA chain and the like) is produced. A voltage is applied between the second electrode 205a within the chamber 203 and the independent first electrode 205b being in contact with the liquid column. By application of the voltage, an electric field is generated around the nano-pore, and the biological molecule receives an electrostatic force by the electric field. By the electrostatic force, the biological molecule passes through the nano-pore. When the biological molecule passes through the nano-pore, the nano-pore is partially blocked by the biological molecule, the resistance value of the nano-pore changes, and therefore the current value detected between the both electrodes 205a, and 205b changes. From the change amount of the current value, the structure of the biological molecule is analyzed. By the processes described above, formation of the nano-pore and analysis of the biological sample are achieved.

### Third Embodiment

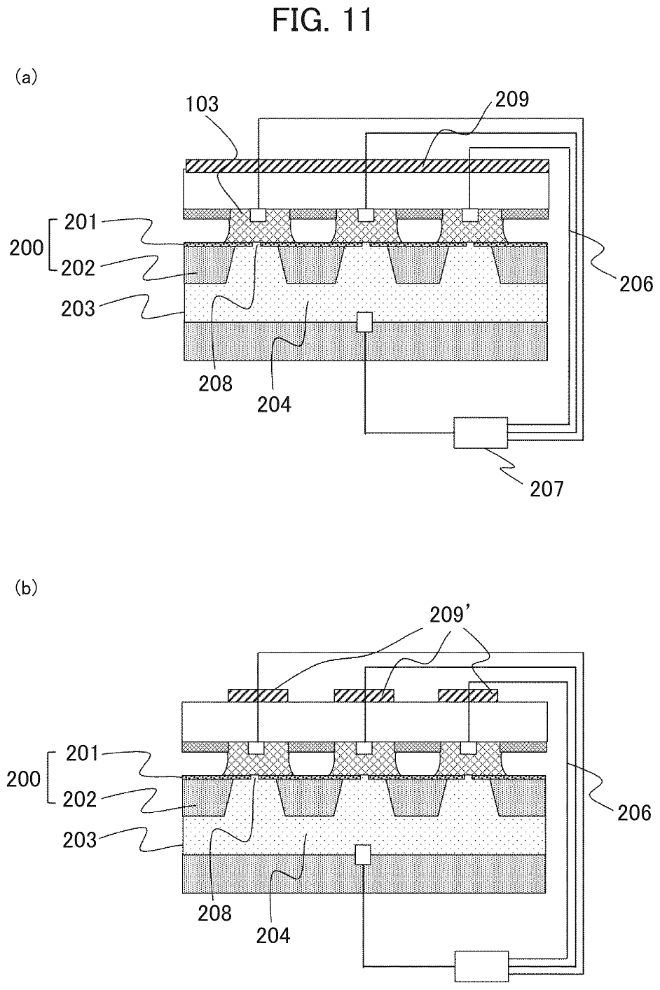

An embodiment combining a liquid column separation device and a temperature control mechanism will be shown. FIG. 11 (a) illustrates a device where a temperature control mechanism 209 such as a Peltier element and a heat block capable of controlling the temperature cycle is arranged outside the first base material on the basis of the device of the second embodiment. By adding the temperature control mechanism, the liquid column containing the biological molecule formed by the liquid column separation device 100 can be temperature-controlled, and it is possible to quickly heat and cool three temperature range required for a PCR reaction (Polymerase Chain Reaction), to maintain a given temperature, and to execute repetition to a designated number of times. Also, as a device illustrated in FIG. 11 (b), temperature control may be executed for each liquid column by dividing a temperature control mechanism 209′ for each hydrophilic well of the first base material. By using the device of the present embodiment, a gene amplified by a PCR reaction can be measured directly by the nano-pore.

Next, a concrete example of the given temperature and the designated number of times described above will be shown. Each cycle of synthesis of DNA by PCR is configured of three steps of denaturation, annealing, and extension. By repeating the PCR cycle by these three steps by several number of times, the target DNA sequence is synthesized. Here, all of the property of DNA polymerase, the kind of PCR buffer solution, and complexity of template DNA affect setup of these reaction conditions.

An example of the PCR reaction is shown below.
(0) First denaturation: 95° C., 5 min. By heating, the double-stranded DNA of the template is separated and becomes a single-strand.
(1) Denaturation: 95° C., 30 sec.
(2) Annealing: 60° C., 60 sec. The primer joins the template.
(3) Extension: 72° C., 60 sec. The DNA extends from the primer portion joined by DNA polymerase.

By repeating three steps of (1), (2), and (3) by 40 cycles, the target of the object region increases exponentially.

The present invention is not limited to the above-described embodiments, and further includes various modifications. For example, the above-described embodiments have been described in detail in order to facilitate the much better understanding of the present invention, and the present invention is not necessarily limited to those including all of the described configurations.

### LIST OF REFERENCE SIGNS

100: liquid column array device, 101: first base material, 102: second base material, 103, 103′: hydrophilic region, 104, 104′: hydrophobic region, 105: peripheral member, 106: filling port, 107: discharge port, 108: tube, 109: liquid feed unit, 110: first solution, 111: second solution, 112: liquid column, 113: liquid droplet, 114, 114′: resistive element, 200: nano-pore device, 201: membrane, 202: substrate with membrane, 203: chamber, 204: electrolyte solution, 205a: electrode, 205b: electrode, 206: wiring, 207: power supply and control/detection data acquisition unit, 208: pore, 209, 209′: temperature control mechanism,
D: representative length of hydrophilic region
H, H′: distance between hydrophilic surface of first base material and second base material
A: cross-sectional aspect ratio

1. A liquid column separation device, comprising:
a first base material;
a second base material disposed to oppose the first base material at a predetermined distance; and
a liquid feed unit feeding two or more fluids to a gap between the first base material and the second base material, wherein
a surface of the first base material opposing the second base material has a pattern of arraying a plurality of hydrophilic regions having hydrophilicity in a hydrophobic region having hydrophobicity, and representative length of the hydrophilic region is larger than the predetermined distance, and
a liquid column that is in contact with the first base material and the second base material is produced by making two or more immiscible fluids flow between the first base material and the second base material by the liquid feed unit.

2. The liquid column separation device according to claim 1, wherein
a distance between the hydrophilic region of the first base material and the second base material is shorter than a distance between the hydrophobic region of the first base material and the second base material.

3. The liquid column separation device according to claim 1, wherein
a column-like object connecting the first base material and the second base material is disposed between the optional neighboring hydrophilic regions of the first base material.

4. The liquid column separation device according to claim 1, wherein
the first base material includes a plurality of independent electrodes disposed within the hydrophilic region.

5. The liquid column separation device according to claim 1, wherein
the second base material includes a hydrophilic region at a position opposing the hydrophilic region of the first base material.

6. The liquid column separation device according to claim 1, wherein
the hydrophilic region of the first base material is of silicon oxide or glass.

7. The liquid column separation device according to claim 1, wherein
a temperature control mechanism is provided in the hydrophilic region of the first base material.

8. A liquid column separation system, comprising:
a liquid column separation device that includes a first base material, a second base material disposed to oppose the first base material at a predetermined distance, and a liquid feed unit feeding two or more fluids to a gap between the first base material and the second base material, a surface of the first base material opposing the second base material having a pattern of arraying a plurality of hydrophilic regions having hydrophilicity in a hydrophobic region having hydrophobicity, representative length of the hydrophilic region being larger than the predetermined distance, a liquid column that is in contact with the first base material and the second base material being produced by making two or more immiscible fluids flow between the first base material and the second base material by the liquid feed unit, the first base material including an electrically independent first electrode in the hydrophilic region, the second base material including a membrane that includes a nano-pore that is in contact with the liquid column;
a chamber including an electrolyte solution that is in contact with the membrane;
a second electrode that is in contact with the chamber;
a measuring unit connected to the second electrode; and
a control unit controlling voltage applied to both electrodes according to a measurement result of the measuring unit, wherein
a biological molecule is introduced to the liquid column and is made to pass through the nano-pore, temporal change of ion current flowing between the both electrodes is measured, thereby passing through of the biological molecule is detected, and a structural characteristic of the biological molecule is analyzed.

9. The liquid column separation system according to claim 8, wherein
distance of the hydrophilic region of the first base material from the second base material is shorter than distance of the hydrophobic region of the first base material from the second base material.

10. The liquid column separation system according to claim 8, wherein
a column-like object connecting the first base material and the second base material is disposed between the optional neighboring hydrophilic regions of the first base material.

11. A liquid column separation method of producing a liquid column being in contact with a first base material and a second base material by making two or more immiscible fluids flow to a gap between the first base material and the second base material of a liquid column separation device by a liquid feed unit, the liquid column separation device being configured to include: the first base material, the second base material disposed to oppose the first base material at a predetermined distance; and the liquid feed unit feeding two or more fluids to a gap between the first base material and the second base material, a surface of the first base material opposing the second base material having a pattern of arraying a plurality of hydrophilic regions having hydrophilicity in a hydrophobic region having hydrophobicity, representative length of the hydrophilic region being larger than the predetermined distance.

12. The liquid column separation method according to claim 11, wherein
a distance between the hydrophilic region of the first base material and the second base material is shorter than a distance between the hydrophobic region of the first base material and the second base material.
